(19) United States
(12) Patent Application Publication
(10) Pub. No.: US 2014/0277035 A1
(43) Pub. Date: Sep. 18, 2014

(54) SYSTEM AND METHOD FOR R-MODE IMAGING AND TREATMENT PLANNING

(71) Applicant: SonaCare Medical, LLC, Charlotte, NC (US)

(72) Inventors: John Strait, Pittsburgh, PA (US); Roy Carlson, Indianapolis, IN (US); Clint Weis, Indianapolis, IN (US)

(73) Assignee: SonaCare Medical, LLC, Charlotte, NC (US)

(21) Appl. No.: 14/212,239

(22) Filed: Mar. 14, 2014

Related U.S. Application Data
(60) Provisional application No. 61/782,566, filed on Mar. 14, 2013.

(51) Int. Cl. A61B 17/32

(52) U.S. Cl. CPC A61B 17/320068
USPC 606/169

(57) ABSTRACT

System and computer-implemented method for operating an ultrasound transducer and generating an R-Mode image from at least a set of images of a region of interest and generating a treatment plan, wherein the treatment plan can be automatically updated in view of defined constraints or manually modified by the operator.

### RELATED APPLICATION DATA

This application claims priority to U.S. Provisional Patent Application No. 61/782,566, filed Mar. 14, 2013, which is hereby incorporated by reference in its entirety. This application further incorporates by reference the disclosure of U.S. Pat. No. 8,235,902 as if fully set forth herein.

### BACKGROUND

Ultrasound devices are commonly used in imaging and therapeutic applications. Ultrasound energy may be directed inside a patient during the course of a therapeutic intervention to image a region of interest, and the reflected energy may be displayed and measured. In this respect, there are a plurality of ultrasound imaging modes that may be used, and each for a given set of one or more circumstances. For example, certain technical features of an ultrasound imaging device may cause constraints on the manner in which reflected energy is detected and processed, or, alternatively, clinical requirements may necessitate use of certain ultrasound device configurations. The imaging modes, as may be appropriate in a given situation, include A-Mode (amplitude mode), wherein a single transducer scans across a line through the region of interest such that the reflected energy (echoes or backscatter) may be plotted as a function of depth; B-Mode (brightness mode or 2-D mode), wherein a linear array of transducers scans a plane through the body such that the region of interest is viewed as a two dimensional image on a display; and C-Mode, wherein the data from B-Mode is displayed as if on a plane normal to a B-Mode image.

Ultrasound energy may also be used during the course of a therapeutic intervention to plan the intervention itself. Interventions in this context may include surgical excision, deposition of thermal energy for ablation or destruction of the (or a part of the) region of interest, and deposition of ionizing energy to destroy the region over time. Planning may involve determining the best path for the intervention procedure, the amount and location of energy or radiation to be deposited in the region of interest, and the best way to deliver energy or radiation in order to avoid regions that are outside the scope of the region to be treated (i.e., outside the region of interest).

Among the technologies being considered or developed or currently deployed for use in treating abnormalities of human and animal tissue is high intensity focused ultrasound (HIFU). Therapeutic HIFU devices utilize ultrasound transducers such that energy is focused so as to deliver a generally thermal or cavitational dosage to a small well-defined location at a fixed distance from the transducer surface. Ultrasound may be used to plan HIFU treatments.

The Sonablate® device manufactured by SonaCare Medical, LLC utilizes ultrasound energy to image and define the region of a prostate that is to be ablated. Typically, ultrasound images are displayed as a combination of orthogonal, coronal, and sagittal slices through the region of interest. The region to be treated is then outlined on the generated ultrasound images and the user of the device is provided information to shape the treatment to conform to the generated outline.

Referring to FIG. 1, a portion of the Sonablate® device 100 is shown, wherein a probe body 101 houses a HIFU transducer 102 and the HIFU transducer 102, which operates in an x, y, z coordinate system. The focal zone of the transducer 102 may be described by its anterior and posterior radii, respectively, rₐ and rₚ. A plurality of focal zones may be used to cover the entire region of interest if the region is thicker than a single zone. Referring now to FIG. 2, as is to be appreciated by those skilled in the art, the positions and sizes of the focal zones can be controlled by changing the location of the HIFU transducer 102 or by changing the focal length of the HIFU transducer 102, or by a combination thereof. Within each treatment zone, therapeutic energy may be delivered by a collection of treatment shots 103 (see FIG. 1), each of which has a volumetric extent.

### BRIEF DESCRIPTION OF THE DRAWINGS

FIG. 1 shows an ultrasound transducer, probe and related focal zone according to the prior art.

FIG. 2 shows various focal zones of an ultrasound transducer over a region of interest according to the prior art.

FIG. 3 shows standard ultrasound imaging geometry according to the prior art.

FIG. 4 shows the geometry of an ultrasound transducer operating in a therapy mode according to the prior art.

FIG. 5 shows a probe coordinate system for an ultrasound transducer configuration according to the prior art.

FIG. 6 shows an R-Mode coordinate system according to an embodiment of the present disclosure.

FIG. 7 shows an R-Mode image designated into rectangular segments according to an embodiment of the present disclosure.

FIG. 8 shows an intersection between a convex, three dimensional treatment region and a treatment zone placed to cover the anterior region according to an embodiment of the present disclosure.

FIG. 9 shows an axial cross-section of an ultrasound transducer configuration oriented to a treatment region according to an embodiment of the present disclosure.

FIG. 10 shows a sagittal cross-section of an ultrasound transducer configuration oriented to a treatment region according to an embodiment of the present disclosure.

FIG. 11 shows an R-Mode surface according to an embodiment of the present disclosure.

FIG. 12 shows an R-Mode surface that has received treatment shots in the treatment region according to an embodiment of the present disclosure.

FIG. 13 shows an R-Mode surface that has received treatment shots in the treatment region according to an embodiment of the present disclosure.

FIG. 14 shows an R-Mode surface that has received treatment shots in the treatment region according to an embodiment of the present disclosure.

FIG. 15 shows various shapes that can be configured according to an embodiment of the present disclosure.

FIG. 16 shows various cross-sections and an interface that can be provided on a display according to an embodiment of the present disclosure.

FIG. 17 shows crosshairs indicating the intersection between cross-sectional views according to an embodiment of the present disclosure.

FIG. 18 shows a three-dimensional rendering for display to an operator according to an embodiment of the present disclosure.

FIG. 19 shows a human computer interface for generating and modifying a treatment plan according to an embodiment of the present disclosure.

FIG. 20 shows a polygonal region of interest as displayed on an interface according to an embodiment of the present disclosure.

FIG. 21 shows a freeform region of interest as displayed on an interface according to an embodiment of the present disclosure.

FIG. 22 shows an arctangular region of interest as displayed on an interface according to an embodiment of the present disclosure.

FIG. 23 shows a rectangular region of interest as displayed on an interface according to an embodiment of the present disclosure.

FIG. 24 shows a rectangular region of interest as displayed on an interface according to an embodiment of the present disclosure.

FIG. 25 shows a treatment plan as displayed on an interface according to an embodiment of the present disclosure.

FIG. 26 shows a treatment plan as displayed on an interface according to an embodiment of the present disclosure.

FIG. 27 shows a treatment plan as displayed on an interface according to an embodiment of the present disclosure.

FIG. 28 shows a treatment plan as displayed on an interface according to an embodiment of the present disclosure.

FIG. 29 shows a treatment plan as displayed on an interface according to an embodiment of the present disclosure.

FIG. 30 shows a treatment plan as displayed on an interface according to an embodiment of the present disclosure.

FIG. 31 shows an informational overlay displayed on an interface according to an embodiment of the present disclosure.

FIG. 32 shows an informational overlay displayed on an interface according to an embodiment of the present disclosure.

FIG. 33 shows an informational overlay displayed on an interface according to an embodiment of the present disclosure.

### DETAILED DESCRIPTION

There is a need in the art to improve the correlation between the ultrasound images used to plan the treatment and the geometry of the actual treatment. That is, referring to the Sonablate® device as an illustrative example, ultrasound images used for treatment planning purposes may be instructive, but less than ideal. The Sonablate® device delivers HIFU therapeutic energy at a fixed radius (focal distance) from the transducer. This results in a focal zone that is an arc sector of defined thickness at a fixed distance from the ultrasound transducer, which is positioned at the origin or center point of the arc. In this case, a number of focal zones may be used to cover the entire region of interest if it is thicker than a single zone. There is, therefore, a potential mismatch between the ultrasound planning images and the geometry of the treatment.

Referring to FIG. 3, and as is to be appreciated by those skilled in the art, a standard ultrasound imaging geometry is shown, wherein planar images are oriented orthogonal to the position of the imaging transducer. In comparison, now referring to FIG. 4, the geometry of HIFU treatment is shown, wherein the treatment is delivered in a curvilinear arc relative to the position of the therapeutic transducer. While the two may be correlated, it requires identification of the target region on multiple orthogonal images which then must be interpreted by the device user, operator or treating physician in order to get a proper geometric representation of the region to be treated. Accordingly, there is a need in the art to display ultrasound images that correspond to the geometry of the treatment delivered by devices that generate curvilinear regions of therapy.

Further, treatment planning is performed currently on axial and sagittal slices through the ultrasound data for the region of interest. In each focal zone, it is necessary to define a collection of individual treatment shots that adequately cover the region to be treated. The treatment plan must also be designed in such a way that tissues outside of the treatment region are spared. As is to be appreciated by those skilled in the art, treating physicians typically define the location of each treatment shot to ensure that relevant constraints are met. By way of example, in the anterior region of the prostate, no more than one-third of each treatment shot should be allowed to extend outside of the prostate. Those skilled in the art will appreciate that different constraints apply in different contexts (e.g., targets other than cancers).

In addition, treatment planning is performed on orthogonal planes of data sliced through the imaging volume. The treatment itself, however, is delivered along an arc sector of defined radial thickness at a fixed distance from the ultrasound transducer. As is to be appreciated by those skilled in the art, the treating physician must mentally relate planar orientation used for planning to the sector orientation used for therapy. It is therefore desirable to have a correlation method that is more reliable and, for example, less subject to human error. Further, because multiple planar slices of data may be required to represent the sector of treatment delivery, planning has to be performed on many images for each sector. This process is potentially time consuming. By way of non-limiting example, a three zone treatment may require several hundred shots of HIFU as defined on as many as 40 or 50 imaging slices of data. A treatment plan such as this can require one hour or more to create. Accordingly, there is a need in the art for an improved system and method to define locations of all treatment shots within a particular focal zone that reduces error and can be performed rapidly.

Various embodiments of the present invention will be described in detail with reference to the drawings. Reference to various embodiments does not limit the scope of the invention, which is limited only by scope of the claims attached hereto. Additionally, any examples set forth in this specification are not intended to be limiting and merely set forth some of the many possible embodiments. Also, it is to be understood that the phraseology and terminology used herein is for the purpose of description and should not be regarded as limiting. The use of “including”, “comprising”, or “having” and variations thereof herein is meant to encompass the items listed thereafter and equivalents thereof as well as additional items. It is to be further appreciated that while various embodiments are described in connection with radiation treatment of tumors, they are not so limited and may be used in connection with other industries and to targets other than cancers. Further, the terms “region of interest” and “treatment region”, and the embodiments disclosed herein should not be interpreted to be limiting between the two; all embodiments, unless otherwise noted, apply equally to a region of interest or a treatment region. In addition, the disclosure is not intended to be limited to treatment or imaging of a single region of interest or treatment region; instead, all techniques apply equally whether a single region or multiple regions are utilized.

The disclosed embodiments are a system and method to generate a linear two dimensional ultrasound image from an ultrasound volumetric data set by constructing a display surface sliced through the imaging data such that it corresponds to the geometry of treatment for an ultrasound transducer 400. This imaging technique is generally referred to herein as R-Mode. In an embodiment, R-Mode imaging eliminates the therapy planning disadvantages stemming from the manner in which conventional orthogonal ultrasound data is displayed. Indeed, instead of displaying the data as orthogonal slices through a three dimensional volume, such data may be displayed following the arc (i.e., radius of the curvature) of the therapy treatment zone. This R-Mode image permits the user to view in a single image the entire (or substantially entire) extent of a region of interest a fixed distance from the therapy device. That is, an embodiment of the present disclosure may allow the images to be registered to, and displayed as, the curvilinear surface of the therapy delivery. In a further embodiment, the pixel intensity values corresponding to a curvilinear slice through a three dimensional data set constructed from a set of 2-D ultrasound images are displayed as a linear plane of intensity values. In a further embodiment, the pixel intensity values corresponding to a curvilinear slice through a three dimensional ultrasound data set are displayed as a linear plane of intensity values. In a further embodiment, the curvilinear slice corresponds to a region of interest at a fixed focal distance from a HIFU treatment device.

As set forth herein, curvilinear images corresponding to the geometry of HIFU treatment systems are referred to as R-Mode images, and may be rendered by a plurality of processes.

In an embodiment, a three dimensional (3-D) ultrasound dataset is generated that provides the ultrasound intensity values by either scanning the patient or target using a three dimensional imaging system or by assembling a series of two dimensional ultrasound images to form a volume, as is known in the art. A further input to the disclosed process is the calculation of the radius of curvature for the image to be generated, R. R may be defined as equal to the focal distance of the HIFU transducer used in the intended treatment, but, alternatively, may be any distance to a region of interest relative to a point of origin. Referring to the three dimensional ultrasound dataset, individual pixel values are known as they are associate with the radius of curvature R in the curved rectangular surface. This dataset may be identified by a controller and displayed as a linear x-y plane of intensity values to the user on a display system. This linear plane represents the intensity values of the transducer (which is positioned at the origin of the coordinate system), and provides information relating to an associated uniform depth as the imaging beam of the transducer sweeps along its arc of travel.

Further, and as is to be appreciated by those skilled in the art, therapeutic ultrasound treatments are delivered along this arc at a distance from the transducer surface that may be specific to the transducer device being used. Accordingly, the pixel intensities that are displayed to the user, as described above, correspond to the region to be treated by the transducer device. The foregoing causes the imaging and treatment delivery surfaces to be co-registered and aligned, such that the region to be treated can be identified on a single reconstructed image corresponding to the specific focal distance of the treatment device to be used. It is to be appreciated that the integrated image described provides the benefit of an accurate and intuitive reference for the user of the transducer, and may be used as an accurate input to treatment plans. In comparison, the absence of the disclosed system and method would require the region to be treated to be identified on all of the orthogonal two dimensional planar images that intersect the region of interest, wherein the treatment plans are based on a plurality of disparate images strung together, instead of a single unified image that corresponds to the actual treatment application zone of the transducer to be used.

In a further embodiment, ultrasound data is sampled at a fixed distance from the ultrasound transducer or probe. In this instance, the R-Mode image may be directly generated from the signal returned by the imaging transducer while sweeping the transducer over all imaging positions. In an embodiment, this may be accomplished by capturing the ultrasound sample at a constant radial distance relative to the transducer or probe.

R-Mode images may be generated by a plurality of mechanisms. Referring to FIG. 5, in an embodiment, a Probe coordinate system relative to the transducer or probe is provided, wherein the z-axis is parallel to the probe's axis and is oriented away from the probe body; the x-axis is perpendicular to the z-axis and is oriented to the left of the probe body; the y-axis is perpendicular to x- and z-axes and oriented up from the probe body in the direction in which ultrasound energy is expressed. The point of origin (0,0,0) is at the central point of the transducer when the transducer is at its initial position in the probe body (i.e., the position closest to the probe body). The radial position of an ultrasound sample relative to the probe axis, rp, may be expressed as rp=\sqrt{x²+y²}. The angular position of an ultrasound sample relative to the Y-axis, θ, may be expressed as

$$θ = {tan}^{- 1} \frac{x}{y} .$$

Referring now to FIG. 6, an R-Mode coordinate system relative to the R-Mode image is provided, wherein the u-axis is horizontal and oriented to the right; the v-axis is vertical and oriented upwards; and the point of origin (x=0, y=0) is at the center of the bottom edge of the image. An R-Mode image may be formed by holding r constant and varying and v. The ultrasound image for the R-Mode image at point (u, v) is derived from the position in the probe space defined by Table 1:

TABLE 1
$θ = \frac{u}{r}$
x = r sin θ
y = r cos θ
z = v

The denominator, r, is required to keep the horizontal image resolution constant at different radial positions, r.

In an embodiment, the R-Mode image may be formed directly from the imaging transducer by using the signal returned by the imaging transducer while sweeping the transducer across all z and θ positions, and by capturing the ultrasound sample at a constant radial distance relative to the transducer or probe. In this case, an ultrasound sample at a particular radial distance may be captured by sampling backscattered RF ultrasound signals at a particular time delay, t, after sending a burst of ultrasound energy at the target. This mechanism may be understood as:

$$t = \begin{matrix}2 ( r - {r}_{0} ) \\ Velocity\end{matrix}$$

Here, velocity is the speed of sound in the medium and r₀ is a radial offset that is introduced to account for the combined effects of the offset of the transducer face from the center of rotation and trigger delay (or advance) to the first digitized signal sample.

In a further embodiment, the R-Mode image may be formed at least partially from volumetric image data. As disclosed above, an R-Mode image may be formed directly from the imaging transducer; however, this is efficient only if the imaging system is optimized for capturing individual planar ultrasound images. In the case that the imaging system is not so optimized, an alternative mechanism may be utilized, wherein multiple ultrasound images are captured in order to build a volume of ultrasound samples. By way of non-limiting example, the transducer may be operated in an imaging sequence to capture a set of images parallel to the x-y plane at equally (or, alternatively, unequally) spaced z positions. In this example, regardless of the imaging sequence utilized, the ultrasound samples are stored into a three dimensional array of ultrasound samples. The three dimensional array may be organized using polar or Cartesian methods.

In the case of a polar volumetric image representation, the ultrasound samples are stored during the imaging process in a three dimensional array indexed by polar coordinates (i.e., r, θ, z). It is desirable to use this process when the ultrasound images are captured as a set of one dimensional (i.e., A-Mode) ultrasound lines without conversion to rectilinear images. After the three dimensional array is filled with ultrasound data, the R-Mode image at any particular radial position, r, may be formed by sampling this polar array at all R-Mode coordinates (u, v), with or without image interpolation. The mechanism for forming the R-Mode image according to the foregoing is set forth in Table 2:

TABLE 2
$θ = \frac{u}{r}$
z = v

In the case of a Cartesian volumetric image representation, the ultrasound samples may be stored during the imaging process into a three dimensional array indexed by Cartesian coordinates (x, y, z). It is desirable to use this process when the ultrasound images are converted or rectilinear images during the imaging process. After the three dimensional array is filled with ultrasound data, the R-Mode image at any radial position, r, may be formed by sampling the Cartesian array at all R-Mode coordinates (u, v), with or without interpolation. A mechanism for deriving the foregoing R-Mode image is described above as set forth in Table 1 above.

In an embodiment, the Cartesian three dimensional array may be sampled by way of one of two mechanisms. First, a continuous sampling method may be utilized, wherein each position in the R-Mode image is independent transformed from the R-Mode coordinate system (u, v) to the Probe coordinate system (x, y, z) utilizing the mechanism set forth above in Table 1. It is disclosed that a more accurate R-Mode image is achieved using this process.

A second sampling method, the segmented sampling method, is further disclosed, wherein the R-Mode image is divided into rectangular segments. Referring to FIG. 7, each rectangular segment may be identified by its four corners in R-Mode coordinates, (u₀,v₀), (u₁,v₁), (u₂,v₂), (u₃,v₃). Each corner of each rectangular segment may be transformed from R-Mode coordinates, (uᵢ,vᵢ), to Probe coordinates, (xᵢ,yᵢ,pᵢ), by using the mechanism set forth in Table 1 in connection with the R-Mode coordinate system. The resulting collection of transformed segments can be used to approximate the surface of the R-Mode arc. After transforming the R-Mode coordinates to the Probe coordinates, each planar segment may be sampled from the three dimensional array of ultrasound samples to form the corresponding segment of the R-Mode image. This process may produce relatively less accurate results in comparison to the continuous sampling method, disclosed above; however, the segmented sampling method may be better suited for implementation with modern graphics hardware and software (e.g., OpenGL®), as is to be appreciated by those skilled in the art. As such, the tradeoff between computational efficiency and spatial inaccuracy can be controlled by varying the number and size of the segments, as may be appropriate.

In a further embodiment, a treatment planning process is disclosed, wherein geometries for treatment plans are generated manually, and, alternatively, automatically (wherein such automatically generated treatment plans satisfy defined constraints that may be expressed as part of a rule set). The disclosed treatment planning process may be used in connection with pre-treatment or post-treatment evaluation processes, as are disclosed herein. Such treatment plans may be utilized to provide descriptions of treatment delivery, and may be superimposed, by electronic means as known in the art, upon ultrasound images.

As described above, treatment planning is performed using planar and/or curvilinear surfaces corresponding to the geometry of a HIFU treatment system. Ultrasound images corresponding to planar surfaces may include “axial” (also referred to as “sector”) images, “sagittal” (also referred to as “transverse” or “linear”) images and coronal images. Ultrasound images corresponding to curvilinear surfaces are referred to as R-Mode images, as disclosed herein.

Treatment planning constraints may apply in each case, and may vary from context to context, as is to be appreciated by those skilled in the art. Referring to FIG. 8 and FIG. 9, the constraints that may be considered include, but are not limited to, placing treatment shots at the anterior of the treatment region, such that no such shots extend more than a defined percentage outside of the treatment region, wherein anterior in this example is defined as the direction away from the HIFU transducer; placing treatment shots in the posterior of the treatment region, such that no such shots extend more than a defined percentage outside of the treatment region, wherein posterior in this example is defined as the direction towards the HIFU transducer; placing treatment shots in such a manner that they do not impinge on regions of interest; and any combination of foregoing constraints.

In an embodiment, a computer program is configured to automatically identify the treatment region and superimpose said treatment region upon the axial, sagittal or R-Mode ultrasound images and/or three dimensional renderings. In an embodiment, the computer program is further configured to automatically place treatment shots to cover the region of interest. Further, treatment planning and delivery may be performed in a single HIFU focal zone or in multiple zones; treatments may be planned for a single treatment region or multiple treatment regions; and treatment regions may represent portions of organs, portions of tumors, or entire organs or entire tumors.

In a further embodiment, the computer program may be configured to superimpose additional information on the axial, sagittal and/or R-Mode ultrasound images and/or three dimensional renderings during treatment planning to provide guidance in the treatment planning process. By way of non-limiting example, Doppler ultrasound information may be displayed to aid the user or treating physician to identify the locations of neurovascular bundles in the proximity of the prostate gland; and/or images produced by other imaging modalities (e.g., MRI or CT imaging) may be superimposed upon the axial, sagittal and/or R-Mode geometry; and/or anatomical structure information derived from other software applications or sources may be superimposed (e.g., DICOM RT structures). Further, additional information may be superimposed upon the above-noted images to aid in the evaluation of results of treatment during and/or after treatment delivery, including, but not limited to, information derived from ultrasound RF data captured before and after delivery at a treatment shot, and such information may be interpreted in order to detect changes to the tissue. This information may be displayed in a segmented fashion consistent with the segmented way that the treatment shots are displayed.

In a further embodiment, the treatment region may be manually identified by a human operator on images corresponding to the geometry of treatment and/or planar ultrasound images. Further, individual treatment shots may be manually added or removed by a human operator on images corresponding to the geometry of treatment and/or planar ultrasound images. The treatment region may be manually identified by a human operator based upon images produced by other imaging modalities (e.g., MRI or CT imaging) using images corresponding to the geometry of treatment and/or planar image geometries. A treatment region may also be automatically identified, but not superimposed on the ultrasound images. Additional information prior, during, and/or after treatment delivery is displayed using images corresponding to the geometry of treatment and/or planar image geometries but is not superimposed upon the ultrasound images.

As discussed above, a Probe coordinate system relative to the transducer or probe is provided having an origin point (x=0, y=0, z=0), wherein the origin is the central point of the transducer when it is at the position closest to the probe. A three dimensional curvilinear surface (x, y, z) at radius r used by treatment planning may be defined by parametric equations with parameters (u, v) as set forth in Table 1. Referring to FIG. 8, an intersection between a convex, three dimensional treatment region 802 and a treatment zone placed to cover the anterior region is disclosed. Referring now to FIG. 9, in an axial cross-section, the intersection described will taper from posterior to anterior if the three dimensional treatment region is convex, wherein a HIFU transducer 901 having posterior and anterior radii, 902, 903, a fixed radius, rₓ, 904, a treatment region 905, an R-Mode surface 906, a focal zone 907 and a single treatment shot 908 are disclosed. Referring now to FIG. 10, in a sagittal cross-section, the intersection described will taper from posterior to anterior, wherein a HIFU transducer 1001 having posterior and anterior radii, 1002, 1003, a fixed radius, rₓ, 1004, a treatment region 1005, an R-Mode surface 1006, a focal zone 1007 and a single treatment shot 1008 are disclosed.

In comparison to the foregoing, referring to FIG. 11, the intersection of the treatment region with an R-Mode surface is described, wherein an R-Mode surface 1101, treatment region 1102, single treatment shot 1103, and tissue outside of the treatment region 1104 is disclosed. The R-Mode surface 1101 is placed at radius

$${r}_{x} = {r}_{p} + x \frac{( {r}_{a} - {r}_{p} )}{100} ,$$

which may be understood as placing the surface at x %, wherein “x” is a variable, between the posterior and anterior radii as described in FIG. 9 and FIG. 10.

As illustrated in FIG. 12, an R-Mode surface 1201 is disclosed, wherein filling the treatment region 1202 creates a treatment plan that covers the treatment region with treatment shots 1203 that do not extend more than x %, wherein “x” is a variable, to tissue outside of the treatment region 1204. In an embodiment, the treatment region may be provided on a display to the user as an R-Mode ultrasound image. Alternatively, it may be displayed in an outline format using the R-Mode image geometry disclosed herein, with or without the ultrasound image. In each case, treatment shots 1203 may be displayed or superimposed upon any displayed images.

The disclosed treatment plan for filling the treatment region with treatment shots may be varied by the choice of radius, rₓ, and by selected constraints as may be appropriate in a given condition. By way of non-limiting example, in an embodiment, a conservative treatment plan may be formulated, wherein no portion of any treatment shot 1203 extends outside of the treatment region 1202 onto the R-Mode surface 1201. Alternatively, referring to FIG. 13, the treatment region 1301 may be filled more aggressively such that treatment shots 1302 are included, for example, even if any portion intersects with the treatment volume. Alternatively, referring to FIG. 14, the treatment region 1401 may be filled more moderately such that treatment shots 1402 are included, for example, if its center intersects with the treatment volume. In an embodiment, the treatment region may be provided to a user via a display as an R-Mode or planar ultrasound image. Alternatively, it may be displayed in an outline format using the R-Mode or planar image geometry, with or without the ultrasound image. Treatment shots may be displayed superimposed upon any display or image, and may be added or removed on any such displays or images.

In a further embodiment, treatment shots may be added or removed manually by the human operator. The operator may identify or otherwise select one or more individual shots by selecting them using a pointing device, such as a mouse, or other interface known in the art. The operator may also identify a grouping of treatment shots by drawing a geometric figure around the shots using the display and interface. Geometric figures may include, but are not limited to, rectangles 1502, so-called arctangles 1501, regular polygons 1504, irregular polygons 1503 and freeform shapes 1505, e.g., as shown in FIG. 15. It is to be appreciated that arctangles are particularly appropriate in the context of axial cross-sections, e.g., as shown in FIG. 9.

Individual shots may be added or removed immediately upon selection of an individual shot, or the shot may be toggled, i.e., added if not present or removed if present. Alternatively, the operator may select the shot and then operate a separate control interface to indicate whether the shot should be added, removed or toggled. In an embodiment, a group of shots may be added or removed immediately upon completion of the drawing of a geometric figure around the shots, or the shots included in the defined geometric figure may be toggled. Alternatively, the operator may draw the geometric figure and then operate a separate control interface to indicate whether shots should be added, removed or toggled. Various toggling rules may be applied; for example, each shot within the geometric figure may be toggled independently. Further, all shots within the region may be added or removed as a group based upon a defined rule. By way of example, shots may be added (in the case that the first selection point when drawing the geometric figure was initially empty), or may be removed (if it was initially filled).

In a further embodiment, a computer interface is disclosed for displaying and/or controlling one or more of the treatment planning mechanisms disclosed herein. By way of non-limiting example, and referring now to FIG. 16, an interface display is provided comprising an axial (or sector) cross section 1601, a sagittal (or linear or transverse) cross-section 1602, an R-Mode cross-section 1603, a three dimensional rendering 1604, and a set of planning controls 1605. Each of the disclosed cross-sectional views represents an ultrasound image showing tissue inside and outside of the treatment region, wherein crosshairs indicate the intersections with the other two cross-sectional views, as shown in FIG. 17. In addition, each cross-sectional view may have two associated slide controls, as are known in the art, that are used by the operator to adjust the positions of the other two cross-sectional views. Referring to FIG. 17, an axial view is shown; however, as is to be appreciated by those skilled in the art, the sagittal and R-Mode cross-sectional views have corresponding items. FIG. 17 shows crosshairs indicating the sagittal position 1701, crosshairs indicating R-Mode position 1702, tissue outside of the treatment region 1703, the treatment region 1704, a slider configured to control the sagittal position 1705, and a slide configured to control the R-Mode position 1706.

In a further embodiment, a three dimensional rendering is provided that shows a stylized representation of the HIFU transducer, a display of ultrasound and/or anatomical data and three surfaces that indicate the locations of the three cross-sectional views. The display of the ultrasound data in the three dimensional rendering may take several forms, each of which may be controlled by the operator. The ultrasound and/or anatomical data may be shown in an ultrasound “cloud” image, as a stylized representation of the treatment region, or a combination of the two. In addition, the planar or curvilinear ultrasound images shown in the cross-sectional views may be displayed in a perspective view relative to the corresponding surfaces that indicate the locations of the three cross-sectional views. As shown in FIG. 18, an exemplary three dimensional rendering is disclosed, comprising a HIFU transducer 1801, a location of an R-Mode cross-section 1802, a location of sagittal cross-section 1803, a location of an axial cross-section 1804, a location of ultrasound and/or anatomical data 1805.

In a further embodiment, the creation of a treatment plan is disclosed, wherein an operator indicates the locations of all treatment shots that will receive HIFU energy, and the treatment plan may be edited by way of a series of steps that either add or remove treatment shots from the treatment plan. In each step, the operator may indicate the boundaries of a region of interest and/or indicate whether to add or remove treatment shots corresponding to the region of interest. Various mechanisms for such selection and designation have been disclosed herein.

In a further embodiment, treatment planning controls are provided, FIG. 19. The treatment planning controls provide buttons or a digital interface (as are known in the art) to edit the treatment plan. Further outlining and drawing tools are known in the art, and such tools may be used in combination with what is disclosed here, or an alternative thereto. A polygon mode interaction 1901 is provided, wherein the operator is enabled to draw a polygon on the axial, sagittal or R-Mode cross-sectional view; for example, as shown in FIG. 20. A polygon may be drawn by using an interface to select each vertex 2001 of the polygon. As the operator adds each vertex 2001, the computer program may draw a line segment 2002 connecting the previous vertex and the currently added vertex, a completed polygon defining a polygonal region of interest 2003. The operator may further move a previously added vertex in the segment between two existing vertices by click-dragging, or as may be appropriate with the interface. A vertex may be removed, for example, by double clicking it. Once the polygon 2003 is complete, the operator may select the Add Shots interface 1913, or the Remove Shots interface 1914 to add/remove shots from the region.

A further freeform mode interaction 1902 is provided, wherein the operator may draw a freeform region 2101 on the axial, sagittal or R-Mode cross-sectional view. To the draw the region, the operator may use any of the human computer interfaces described above, or others as are known in the art. Once the freeform region 2101 is complete, the operator may select the Add Shots interface 1913, or the Remove Shots interface 1914 to add/remove shots from the region.

A further arctangle/rectangle mode interaction 1903 is provided, wherein the operator may draw an arctangular region 2201 on the axial cross-sectional view or draw a rectangular view on the sagittal 2301 or R-Mode cross-sectional view 2401. To draw an arctangle, the operator may click on the treatment shot at the left or right of the region 2202, 2203 and drag the treatment shot to the right or left end, respectively. Alternatively, the operator may click and release in a single position to create an arctangle that surrounds a single treatment shot. The operator need not indicate the height of the arctangle, as it may, in certain embodiments, be automatically be set to the height of the focal zone. To draw a rectangle on the sagittal cross-section, the operator may, for example, click on the treatment shot at the left or right of the region 2302, 2303 and drag the treatment shot to the right or left end, respectively. Alternatively, the operator may, for example, click and release in a single position to create a rectangle that surrounds a single treatment shot. The operator need not indicate the height of the rectangle, as it may, in certain embodiments, be automatically be set to the height of the focal zone. To draw a rectangle on the R-Mode cross-section, the operator may click on the treatment shot at one corner of the region 2402 and rag to the treatment shot at the opposite side 2403. Alternatively, the operator may click and release in a single position to create a rectangle that surrounds a single treatment shot. The operator must indicate both width and height of the rectangle because the rectangle is the R-Mode surface of the focal zone. The drawing region on the R-Mode cross-sectional view creates an arctangular prism, i.e., the shape is arctangular in axial cross-section and extends to multiple treatment shots in the sagittal cross-section. Upon completion of the arctangle or rectangle drawing, the software program may be configured to display the intersection of the specified treatment region with the other two cross-sectional views. For example, after drawing on the axial or sagittal cross-section, the intersection with the R-Mode cross-section is a horizontal or vertical line of treatment shots and the intersection with the other planar cross-section (sagittal or axial) may be either a single treatment shot or may be empty (in the case that the region does not intersect with the cross-sectional view). An operator may choose to utilize the Add Shots interface 1913 or Remove Shots interface 1914 as needed, after the completion of the drawing, in order to add or remove treatment shots from the region on the applicable cross-section, and may, alternatively, continue to make edits to the figure boundaries. In all cases, the boundaries of a region may be manipulated by way of mouse or other interface interactions for any of the three cross-sectional views to enlarge or reduce the region in any direction; thereby, allowing the operator to define a region that has a defined extent on any or all of the views.

The operator may further utilize, without limitation, the following control interactions: Reduce Arctangle interaction 1905 and Enlarge Arctangle interaction 1906 to remove or add, respectively, a single row of treatment shots either from the left or right edge in an axial cross-section; Move Arctangle Left interaction 1909 and Move Arctange Right interaction 1910 in order to move the arctangular region to the left/right, respectively, in an axial cross-section by adding a single row of treatment shots to the left/right edge and removing one from the right/left edge; Reduce Rectangle interaction 1907 and Enlarge Rectangle interaction 1908 in order to remove/add, respectively, a single row of treatment shots from the left or right edge in an axial cross-section; Move Rectangle Left interaction 1911 and Move Rectangle Right interaction 1912 in order to move the rectangular region to the left/right, respectively, in sagittal cross-section by adding a single row of treatment shots to the left/right edge and removing one from the right/left edge.

An Immediate Mode interaction 1904 is further disclosed, wherein rapid changes to the treatment plan may be implemented. The disclosed interaction reduces the number of operator interactions required for editing a panel. For example, in Immediate Mode, the operator may draw an arctangular region on the axial cross-sectional view or draw a rectangular region on the sagittal or R-Mode cross-sectional view, but does not use the Add Shots 1913 or Remove Shots 1914 interactions. The editing operation of adding or removing shots is, instead, performed immediately when the operator releases the mouse button, or other interface used. Further, if the initial position chosen by the operator was empty, treatment shots are added to the region. Conversely, if the initial position selected by the operator was full or contained a treatment shot, then the treatment shots are removed from the region. As is to be appreciated by those skilled in the art, it is advantageous to an operator to be able to add and remove individual shots quickly and with as few interface interactions possible, e.g., a single click and release interaction. Undo Edit interaction 1915 and Redo Edit interaction 1916 are further disclosed, wherein multi-step undo/redo functions may be carried out, and wherein, optionally, the operator may be provided with an opportunity to select the series of steps that are to be undone or replicated.

The disclosed collection of treatment planning tools are advantageous to operators and practitioners because they enable rapid planning of treatment shots that are best suited for typical treatment planning goals. Without intending to limit the scope of this disclosure, an exemplary embodiment is provided wherein a treatment plan in the anterior zone of the prostate is required, wherein no treatment shot extends more than 33% outside of the prostate and avoids nearby critical structures, e.g., the pubic bone. To generate the plan, the operator must first adjust the HIFU transducer to the correct position for treating the anterior focal zone; adjust the position of the horizontal slider above the axial image such that the sagittal cross-sectional view displays the midline of the prostate gland; adjust the position of the horizontal slider over the sagittal image such that the vertical crosshairs intersect the prostate image at its highest point in the sagittal view, as shown in FIG. 25, wherein the prostate gland 2501, the rectal wall 2502 and the pubic bone 2503 are shown, thereby, the calibration causing the largest axial cross-section of the prostate 2501, 2601 to be displayed in the axial cross-sectional view; adjust the position of the vertical slider on the right of either the axial or sagittal view such that the R-Mode crosshair is 33% below the top of the focal zone, i.e., 67% above the bottom of the zone, as shown in FIG. 26 and FIG. 27, wherein the prostate 2601, 2701, the pubic bone 2702, rectal wall 2602, the top and bottom of the transducer's focal zone 2603, and the crosshair at a position that is 33% below the top of the focal zone 2604 are shown.

Further, the operator may select the Polygon Mode interaction 1901, and draw a region coincident with the boundary of the prostate as visible on the R-Mode cross-sectional view, as shown in FIG. 28, wherein the polygonal region of interest 2801 is shown. The operator may then select the Add Shots interaction 1913 to cause the computer program to automatically fill the region with treatment shots according to a set of rules that include, without limitation, the treatment shots are included only if their center points are enclosed by the defined polygon. The anterior focal zone of the prostate is therefore caused to be covered with treatment shots; none of which extend more than 33% outside of the prostate, as shown in FIG. 29, wherein treatment shots that have a center point enclosed by the defined polygon 2901 are shown. In this example, some treatment shots may be too close to critical regions, e.g., the pubic bone 3001, as shown in FIG. 30, wherein the rectal wall 3002, prostate gland 3003, treatment shots 3004, and treatment shots extending into the pubic bone 3001 and should, therefore, be removed 3005 are shown. The operator may select the Immediate Mode interaction 1904, and select a treatment shot on any cross-sectional view to remove individual treatment shots that are too close to critical structures 3005.

During the disclosed planning process, additional information may be overlaid upon the ultrasound image data in any of the displayed geometries, including the three dimensional view, to aid the operator in generating the treatment plan. For example, if Doppler ultrasound data is available, it may be displayed as a color tint or outline overlaying the grayscale ultrasound images. If, for example, images produced by other imaging modalities (e.g., MRI or CT imaging) are available, the ultrasound image data may be replaced by the other images, and, further, if anatomical structure information is available (e.g., DICOM RT structures), it may be displayed as a color tint or as outlines overlaying the grayscale images. Referring to FIG. 31, an exemplary overlay is provided, wherein the rectal wall 3101, the prostate gland 3102 and a color tint overlay indicating the positions of neurovascular bundles as determined from Doppler imaging 3103 is shown.

As the delivery of HIFU energy to the treatment region proceeds and after treatment is completed, additional information may be superimposed upon the axial, sagittal and/or R-Mode ultrasound images and/or three dimensional renderings to help evaluate the results of the treatment. The information may be displayed in a segmented fashion, thereby correlating the results of the treatment shot with the position of the treatment shot itself. By way of further example, information derived from ultrasound RF data captured before and after delivery at a treatment shot may be interpreted in order to detect changes to the tissue. The information may be displayed overlaid upon the ultrasound images or in a schematic representation for a summary view. Referring to FIG. 32, an exemplary overlay is described, wherein treatment shots showing tissue change information after three shots have been delivered 3201 is shown. Referring now to FIG. 33, an exemplary summary view is described, wherein treatment shots showing tissue change information after a portion of the treatment has been delivered 3301 and no ultrasound images 3302 are shown. During treatment delivery, these exemplary displays as have been disclosed herein track the treatment and provide a visual indication of the treatment progress and the remaining treatment. At treatment completion, the displays will show the entire treatment delivery in a single interface and unified representation.

As disclosed herein, various interfaces, displays (referring to representations and information presented to an operator by way of display equipment) and information generation mechanisms are disclosed, each of which may be implemented by computer software code recorded on computer readable media and executed by a processor. The various interfaces, displays and information generation mechanisms described herein may be implemented as modules, but such modules need not be discrete devices or code portions. The modules can be segregated or integrated in any manner. As is to be appreciated by those skilled in the art, various computer devices, controllers and/or processors can be used to implement the embodiments, such as servers, PCs, laptop computers, tablets, handheld computing devices, mobile devices or various combinations of such devices. Further, devices specific to HIFU imaging and therapy may be used to implement the embodiments, including, but not limited to, various transducers and imaging products. Further, various interfaces and controls may be used to interact with the displays and information provided to users, such as keyboards, mice, touchscreens, haptic sensing devices, and styluses. Network connections as are known in the art may be provided to provide information connectivity between modules and/or devices, or to incorporate disparate datasets into the embodiments set forth herein.

What is claimed is:

1. A computer system for generating a treatment plan, the system comprising:
an ultrasound transducer capable of operating in an imaging and therapy mode;
a memory, storing computer executable instructions; and
a processor operatively coupled to said memory and configured to execute the instructions to perform the following steps:
receiving a set of images from the ultrasound transducer operating in an imaging mode as oriented to a region of interest;
generating an R-Mode image from the set of images;
generating a treatment plan; and
providing an interface for modifying the treatment plan and monitoring progress of the treatment plan during execution by the ultrasound transducer operating in the therapy mode.

2. The system according to claim 1, wherein the processor provides an R-Mode coordinate system relative to the R-Mode image.

3. The system according to claim 1, wherein the set of images are stored into a three dimensional array indexed by Cartesian coordinates.

4. The system according to claim 1, wherein the treatment plan is generated such that no portion of any treatment shot extends outside of a treatment region onto an R-Mode surface.

5. The system according to claim 1, wherein the treatment plan is filled such that treatment shots are included even if any portion intersects with a treatment volume.

6. The system according to claim 1, wherein the treatment plan is filled such that a treatment shot is included only if a center of the treatment shot intersects with a treatment volume.

7. At least one non-transitory computer readable medium having stored thereon data representing sequences of instructions, which when executed by at least one computing device, cause the at least one computing device to:
receive a plurality of imaging data from an ultrasound transducer operating in an imaging mode;
generate an R-Mode image corresponding to the received imaging data;
generate a treatment plan;
receive modifications to the treatment plan by way of an interface; and
provide updates as to the execution of the treatment plan on a target as carried out by the ultrasound transducer operating in a therapy mode.

8. The computer readable medium of claim 7, further comprising sequences of instructions, which when executed by at least one computing device, cause the at least one computing device to provide an R-Mode coordinate system relative to the R-Mode image.

9. The computer readable medium of claim 7, further comprising sequences of instructions, which when executed by at least one computing device, cause the at least one computing device to store the set of images into a three dimensional array indexed by Cartesian coordinates.

10. The computer readable medium of claim 7, further comprising sequences of instructions, which when executed by at least one computing device, cause the at least one computing device to generate the treatment plan such that no portion of any treatment shot extends outside of a treatment region onto an R-Mode surface.

11. The computer readable medium of claim 7, further comprising sequences of instructions, which when executed by at least one computing device, cause the at least one computing device to generate the treatment plan such that the treatment region is filled with treatment shots, and the treatment shots are included even if any portion intersects with a treatment volume.

12. The computer readable medium of claim 7, further comprising sequences of instructions, which when executed by at least one computing device, cause the at least one computing device to generate the treatment plan such that the treatment region is filled with treatment shot, wherein the treatment shot is included only if a center of the treatment shot intersects with a treatment volume

13. A method executed by one or more computing for generating a treatment plan, the method comprising the steps of:
receiving, by a computer, a set of images for a target region;
generating, by a computer, an R-Mode image for the target region as derived from the received set of images;
generating, by a computer, a treatment plan;
receiving, by a computer, modifications to the treatment plan;
generating, by a computer, status updates for the treatment plan correlating to the treatment of the target region by an ultrasound transducer operating in a therapy mode and executing at least a portion of the treatment plan.

14. The method of claim 13, further comprising generating, by a computer, an R-Mode coordinate system relative to the R-Mode image.

15. The method of claim 13, further comprising storing, by a computer, the set of images into a three dimensional array indexed by Cartesian coordinates.

16. The method of claim 13, further comprising generating, by a computer, the treatment plan such that no portion of any treatment shot extends outside of a treatment region onto an R-Mode surface.

17. The method of claim 13, further comprising generating, by a computer, the treatment plan such that the treatment region is filled with treatment shots, and the treatment shots are included even if any portion intersects with a treatment volume.

18. The method of claim 13, further comprising generating, by a computer, the treatment plan such that the treatment region is filled with treatment shot, wherein the treatment shot is included only if a center of the treatment shot intersects with a treatment volume.
